(19) United States
(12) Patent Application Publication
(10) Pub. No.: US 2023/0113411 A1
(43) Pub. Date: Apr. 13, 2023

(54) FLEXIBLE PRODUCT SEPARATION AND RECOVERY

(71) Applicant: LanzaTech, Inc., Skokie, IL (US)

(72) Inventors: Allan Haiming Gao, Skokie, IL (US); Robert John Conrado, Washington, DC (US); Joss Anton Coombes, Chicago, IL (US); Nicholas Bourdakos, Toronto (CA)

(21) Appl. No.: 17/450,802

(22) Filed: Oct. 13, 2021

(51) Int. Cl. C12P 7/06; C12M 1/00; B01D 3/40; B01D 3/10; B01D 3/14

(52) U.S. Cl. CPC C12P 7/06; C12M 47/10; B01D 3/40; B01D 3/10; B01D 3/143

(57) ABSTRACT

This disclosure is related to a process and apparatus for producing and recovering at least one fermentation product from a fermentation process using a C1-containing gas passed to a fermentation bioreactor, that produces a fermentation broth comprising at least one of a first product stream comprising ethanol and water or a second product stream comprising ethanol, acetone, and water or a third product stream comprising ethanol, acetone, isopropanol, and water. The product is recovered by using a shared product recovery system. Particularly, the shared product recovery system selectively recovers at least one enriched product stream selected from an enriched ethanol stream, an enriched acetone stream, an enriched isopropanol stream or combinations thereof. The shared product recovery system includes at least one of a vacuum distillation unit, a rectification unit, an acetone removal unit, a drying unit, an ethanol-acetone separation unit, an extractive distillation unit or combinations thereof.

### FIELD

This disclosure relates to a flexible method for recovering products from a fermentation broth, where the fermentation broth includes at least one of ethanol, acetone, and isopropanol.

### BACKGROUND

Carbon dioxide (CO₂) accounts for about 76% of global greenhouse gas emissions from human activities, with methane (16%), nitrous oxide (6%), and fluorinated gases (2%) accounting for the balance (United States Environmental Protection Agency). The majority of CO₂ comes from the burning fossil fuels to produce energy, although industrial and forestry practices also emit CO₂ into the atmosphere. Reduction of greenhouse gas emissions, particularly CO₂, is critical to halt the progression of global warming and the accompanying shifts in climate and weather.

It has long been recognized that catalytic processes, such as the Fischer-Tropsch process, may be used to convert gases containing carbon dioxide (CO₂), carbon monoxide (CO), and/or hydrogen (H₂), such as industrial waste gas or syngas or mixtures thereof into a variety of chemicals such as ethanol, acetone, and isopropanol. Syngas can also be converted to various chemicals by the Monsanto process by converting to methanol as a first step. Both Fischer-Tropsch and Methanol synthesis units are optimized at very high capacities. They require well defined feed gas compositions and syngas feed with low impurities to avoid poisoning the catalysts. Fischer-Tropsch process requires complex and costly purification equipment to generate high purity industrial chemicals. Recently, gas fermentation has emerged as an alternative platform for the biological fixation of such gases. C1-fixing microorganisms have been demonstrated to convert gases containing CO₂, CO, and/or H₂ such as industrial waste gas or syngas or mixtures thereof into products such as ethanol and 2,3-butanediol.

In certain instances, fermentation of a C1-containing industrial gas is tailored to produce a specific chemical product such as ethanol, acetone, or isopropanol. However, although production of the particular product is targeted, the fermentation products will contain other components, for example ethanol and acetone or isopropanol and ethanol. Downstream separation and recovery of the particular chemical product requires individually customised separation systems for each chemical product such as ethanol, acetone, and isopropanol.

Accordingly, there exists a need for an integrated recovery system which has the flexibility to recover different chemical product combinations such as ethanol/acetone or isopropanol/ethanol using shared separation and recovery components instead of using customized separation and recovery components for each combination of products.

### BRIEF SUMMARY

In one embodiment, a process for producing and recovering at least one product from a fermentation process comprises introducing a C1-containing gas from a source to a fermentation bioreactor containing at least one C1-fixing microorganism, in a liquid nutrient medium to produce a fermentation broth comprising at least one of a first product stream comprising ethanol and water or a second product stream comprising ethanol, acetone, and water or a third product stream comprising ethanol, acetone, isopropanol, and water, and transferring the fermentation broth from the fermentation bioreactor to a shared product recovery system for selectively recovering at least one enriched product stream selected from an enriched ethanol stream, an enriched acetone stream, an enriched isopropanol stream or combinations thereof.

In another embodiment, the shared product recovery system can comprise at least one of a vacuum distillation unit, a rectification unit, an acetone removal unit, a drying unit, an ethanol-acetone separation unit, an extractive distillation unit or combinations thereof.

In one aspect, the vacuum distillation unit produces an enriched ethanol stream and a product depleted stream from the fermentation broth comprising the first product stream wherein the product depleted stream is returned to the fermentation bioreactor. In another aspect, the vacuum distillation unit produces a concentrated stream enriched in acetone and ethanol and a product depleted stream from the fermentation broth comprising the second product stream wherein the product depleted stream is returned to the fermentation bioreactor. In still another aspect, the vacuum distillation unit produces a concentrated stream enriched in isopropanol, acetone and ethanol and a product depleted stream from the fermentation broth comprising the third product stream wherein the product depleted stream is returned to the fermentation bioreactor.

In yet another embodiment, the C1-fixing microorganism is switched from a C1-fixing microorganism which produces the first product stream to one which produces the second product stream of ethanol, acetone, and water or the third product stream of ethanol, acetone, isopropanol, and water; or from a C1-fixing microorganism which produces the second product stream to one which produces the first product stream or the third product stream; or from a C1-fixing microorganism which produces the third product stream to one which produces the first product stream or the second product stream.

In a further embodiment, a system to recover at least one product from a gas fermentation process comprises (a) a C1-gas fermentation bioreactor in fluid communication with a vacuum distillation unit configured to produce an enriched ethanol stream and a product depleted stream from a first product stream comprising ethanol and water, and (b) a rectification unit in fluid communication with the vacuum distillation unit, the rectification unit being configured to produce an overhead ethanol stream and a bottom water stream.

In another embodiment, a drying unit is in fluid communication with the rectification unit, the drying unit being configured to produce an anhydrous ethanol stream and a purge stream.

In still another embodiment, a system to recover at least one product from a gas fermentation process comprises (a) a C1-gas fermentation bioreactor in fluid communication with a vacuum distillation unit configured to produce a concentrated stream enriched in acetone and ethanol and a product depleted stream from a second product stream comprising ethanol, acetone, and water (b) a rectification unit in fluid communication with the vacuum distillation unit, the rectification unit being configured to produce an overhead stream enriched in acetone and ethanol and a bottom water stream (c) a drying unit in fluid communication with the rectification unit, the drying unit being configured to produce an anhydrous concentrated stream enriched in acetone and ethanol and a purge stream, and (d) an ethanol-acetone separation unit in fluid communication with the drying unit, the ethanol-acetone separation unit being configured to produce an anhydrous acetone stream and an anhydrous ethanol stream.

In yet another embodiment, a system to recover at least one product from a gas fermentation process comprises (a) a C1-gas fermentation bioreactor in fluid communication with a vacuum distillation unit configured to produce a concentrated stream enriched in isopropanol, acetone, and ethanol and a product depleted stream from a third product stream comprising ethanol, acetone, isopropanol, and water (b) an acetone removal unit in fluid communication with the vacuum distillation unit, the acetone removal unit being configured to produce a bottom stream enriched in isopropanol and ethanol and an overhead stream rich in acetone (c) a rectification unit in fluid communication with the acetone removal unit, the rectification unit being configured to produce an overhead stream enriched in isopropanol and ethanol and a bottom water stream from the bottom stream (d) a drying unit in fluid communication with the rectification unit, the drying unit being configured to produce an anhydrous concentrated stream enriched in isopropanol and ethanol and a purge stream and, (e) an extractive distillation unit in fluid communication with the drying unit, the extractive distillation unit being configured to obtain an overhead stream and a distillation bottom stream from distillation of the anhydrous concentrated stream enriched in isopropanol and ethanol in presence of at least one extractive distillation agent.

A further embodiment involves having the extractive distillation unit in fluid communication with a separation column and another separation column, being configured to recover (i) at least a portion of anhydrous ethanol from the overhead stream and at least a portion of anhydrous isopropanol from the distillation bottom stream; or (ii) at least a portion of anhydrous isopropanol from the overhead stream and at least a portion of anhydrous ethanol from the distillation bottom stream.

In still a further embodiment, the acetone removal unit is in further fluid communication with the fermentation bioreactor, the acetone removal unit being configured to recycle the overhead stream to the fermentation bioreactor.

The foregoing and other objects, embodiments and features of the present disclosure will become more apparent from the following detailed description, which proceeds with reference to the accompanying figures.

### BRIEF DESCRIPTION OF THE DRAWINGS

FIG. 1 is a schematic flow diagram showing an overall gas fermentation process including a fermentation bioreactor, and a shared product recovery system in accordance with one aspect of the disclosure.

FIG. 2 is a flow diagram showing details of a vacuum distillation unit, a rectification unit, and a drying unit of the shared product recovery system in accordance with a first aspect of the disclosure.

FIG. 3 is a flow diagram showing details of a vacuum distillation unit, a rectification unit, a drying unit, and an ethanol-acetone separation unit of the shared product recovery system in accordance with a second aspect of the disclosure.

FIG. 4 is a flow diagram showing details of a vacuum distillation unit, an acetone removal unit, a rectification unit, a drying unit, and an extractive distillation unit having separation columns connected therewith of the shared product recovery system in accordance with a third aspect of the disclosure.

### DETAILED DESCRIPTION

In accordance with the disclosure, a flexible separation and recovery process and system downstream of a fermentation bioreactor is able to separate and recover various combinations of chemical products such as ethanol/acetone or isopropanol/ethanol present in the fermentation broth from the bioreactor. The flexible recovery/separation system/process minimizes the number of units which need to be used.

### Definitions

The term “fermentation broth” or “broth” is intended to encompass the mixture of components is a multiphase gas-liquid aqueous mixture containing unreacted feed gas, culture of one or more microorganism, chemical nutrients, and fermentation products such as ethanol, acetone, isopropanol, and combinations thereof. The term microorganism and the term bacteria are used interchangeably throughout the document.

“Nutrient media” or “nutrient medium” is used to describe microbial growth media. Generally, this term refers to a media containing nutrients and other components appropriate for the growth of a microbial culture. The term “nutrient” includes any substance that may be utilized in a metabolic pathway of a microorganism. Exemplary nutrients include, potassium, B vitamins, trace metals, and amino acids.

The term “enriched product stream” is used to represent percent weight concentration of target product in recovered product stream after passing the fermentation broth through a shared product recovery system. For example, the enriched ethanol stream comprises at least 15% or at least 30% or at least 60% or at least 80% or at least 95% or at least 98% ethanol. Similarly the enriched acetone stream comprises at least 14%, or at least 32% or at least 65% or at least 85% or least 95% or at least 99% acetone. The enriched isopropanol stream comprises at least 16%, or at least 33%, or at least 66% or at least 87% or at least 95% or at least 99% isopropanol.

The term “anhydrous stream” is used to represent an “anhydrous ethanol stream” or an “anhydrous acetone stream” or “anhydrous isopropanol stream” comprising weight concentration less than 5% or less than 2% or less than 1% or less than 0.5% or less than 0.2% or less than 0.1% of water.

In an embodiment, the fermentation broth is produced in a “bioreactor”/“fermentation bioreactor”. The term “bioreactor” includes a fermentation device consisting of one or more vessels and/or towers or piping arrangements which includes, the Continuous Stirred Tank Reactor (CSTR), Immobilized Cell Recycle (ICR), Trickle Bed Reactor (TBR), Bubble Column, Gas Lift Fermenter, Static Mixer, a circulated loop reactor, a membrane reactor, such as a Hollow Fibre Membrane Bioreactor (HFM BR) or other vessel or other device suitable for gas-liquid contact. The bioreactor receives a gaseous substrate comprising CO or CO₂ or H₂ or mixtures thereof. The bioreactor may comprise system of multiple reactors (stages) either in parallel or in series. For example, the bioreactor may comprise a first growth reactor which cultures the bacteria and a second fermentation reactor to which output from the growth reactor may be fed and produce most of the fermentation products. In some embodiments, multiple bioreactors in a bioreactor system are placed on top of another to form a stack. A stack of bioreactors improves the throughput of the bioreactor system without significantly increasing demand for land area. In some embodiments, the bioreactors include microbubble bioreactors having mechanisms to substantially improve rate of gas-liquid mass transfer without increasing energy consumption.

The term “inoculation reactor”, “inoculator” and the like includes a fermentation device for establishing and promoting cell growth. The inoculation reactor preferably receives a gaseous substrate comprising CO or CO₂ or H₂ or mixtures thereof. Preferably, the inoculation reactor is a reactor where cell growth is first initiated. In various embodiments, the inoculation reactor is a vessel where previously grown cells are revived. In various embodiments, the cell growth in the inoculation reactor produces an inoculum, which may be transferred to the bioreactor system where the bioreactor promotes production of one or more fermentation product. In certain instances, the inoculation reactor has a reduced volume when compared to the subsequent one or more bioreactor. In some embodiments, the growth reactor in the bioreactor system may be used as inoculation reactor.

The microorganisms in the bioreactor may be modified from a naturally-occurring microorganism. A “parental microorganism” is a microorganism that generates a microorganism of the disclosure. The parental microorganism may be a naturally-occurring microorganism (i.e., a wild-type microorganism) or a microorganism previously modified (i.e., a mutant or recombinant microorganism). The microorganism of the disclosure may be modified to express or overexpress one or more enzymes that were not expressed or overexpressed in the parental microorganism. Similarly, the microorganism of the disclosure may be modified to contain one or more genes that were not contained in the parental microorganism. The microorganism of the disclosure may also be modified to not express or to express lower amounts of one or more enzymes that were expressed in the parental microorganism. In accordance with one embodiment, the parental microorganism is Clostridium autoethanogenum, Clostridium ljungdahlii, or Clostridium ragsdalei. In one embodiment, the parental microorganism is Clostridium autoethanogenum LZ1561 which was deposited on Jun. 7, 2010, with Deutsche Sammlung von Mikroorganismen und Zellkulturen GmbH (DSMZ) located at Inhoffenstraβ 7B, D-38124 Braunschwieg, under the terms of the Budapest Treaty and accorded accession number DSM23693. This strain is described in International Patent Application No. PCT/NZ2011/000144, which is published as WO 2012/015317.

A “C1-fixing microorganism” is a microorganism that produces one or more products from a C1-carbon source. Typically, the microorganism of the disclosure is a C1-fixing bacterium. The “C1-carbon source” refers a one carbon-molecule that serves as a partial or sole carbon source for the microorganism. For example, the C1-carbon source may comprise one or more of CO, CO₂, CH₄, CH₃OH, or CH₂O₂. In an embodiment, the C1-carbon source comprises one or both of CO and CO₂.

The C1-carbon source may be obtained from a waste gas obtained as a byproduct of an industrial process or from another source, such as combustion engine exhaust fumes, biogas, landfill gas, direct air capture, or from electrolysis. A substrate and/or the C1-carbon source may be syngas generated by pyrolysis, torrefaction, or gasification. In other words, carbon in waste material may be recycled by pyrolysis, torrefaction, or gasification to generate syngas which is used as the substrate and/or the C1-carbon source. The substrate and/or the C1-carbon source may be a gas comprising methane, and in certain embodiments the substrate and/or the C1-carbon source may be a non-waste gas.

“Acetogens” are obligately anaerobic bacteria that use “Wood-Ljungdahl” pathway as a (1) mechanism for the reductive synthesis of acetyl-CoA from CO2, (2) terminal electron-accepting energy conserving process, (3) mechanism for the fixation (assimilation) of CO₂ in the synthesis of cell carbon (Drake, Acetogenic Prokaryotes, In: The Prokaryotes, 3^{rd} edition, p. 354, New York, N.Y., 2006). Typically, the microorganism of the disclosure may be an acetogen.

An “ethanologen” is a microorganism capable of producing ethanol. Typically, the microorganism of the disclosure may be an ethanologen.

An “autotroph” is a microorganism capable of growing in absence of organic carbon. Instead, autotrophs use inorganic carbon sources, such as CO and/or CO₂. Typically, the microorganism of the disclosure may be an autotroph.

A “carboxydotroph” is a microorganism capable of utilizing CO as a sole source of carbon and energy. Typically, the microorganism of the disclosure may be a carboxydotroph.

A “native product” is a product produced by a genetically unmodified microorganism. For example, ethanol, acetate, and 2,3-butanediol are native products of Clostridium autoethanogenum, Clostridium ljungdahlii, and Clostridium ragsdalei. A genetically modified microorganism produces a “non-native product” which is not produced by a genetically unmodified microorganism from which the genetically modified microorganism is derived.

A “shared product recovery system” comprises arranged device combinations operated under similar operating conditions for selectively recovering at least one enriched product stream selected from an enriched ethanol stream, an enriched acetone stream, an enriched isopropanol stream or combinations thereof. Accordingly, the shared product recovery system can comprise at least one of a vacuum distillation unit, a rectification unit, an acetone removal unit, a drying unit, an ethanol-acetone separation unit, an extractive distillation unit or combinations thereof, depending on product stream to be recovered.

The term “vacuum distillation unit” is intended to encompass a device for conducting distillation under vacuum wherein the fermentation product being distilled is enclosed at a low pressure to reduce its boiling point. In an embodiment, the vacuum distillation unit includes a separation section. The fermentation product may be sourced from the bioreactor.

The “vacuum distillation unit” recovers one or more “low boiling fermentation product.” A “low boiling fermentation product” is more volatile than water. These products may include, but are not limited to, ethanol, acetone, isopropanol, butanol, ketones, methyl ethyl ketone, 2-butanol, 1-propanol, methyl acetate, ethyl acetate, butanone, 1,3-butadiene, isoprene, and isobutene.

The “separation section” may be composed of any suitable medium providing a large surface area for vapor-liquid contact which increases effectiveness of the vacuum distillation unit. The separation medium is designed to provide a plurality of theoretical distillation stages. In at least one embodiment, the separation medium is a series of distillation trays. In at least one embodiment, the separation medium is composed of at least one packing material. The packing materials may generally include, thin corrugated metal plates or gauzes arranged in a way that force fluid to flow through desired paths in the vacuum distillation unit.

“Distillation trays” or “distillation plates” and the like are intended to encompass plates and/or trays used to encourage a vapor-liquid contact. Tray types include, but are not limited to, sieve tray, valve tray, and bubble cap tray. The sieve tray containing holes for vapor to flow therethrough are used for high capacity situations providing high efficiency at a low cost. Although less expensive, the valve tray containing holes with opening and closing valves have tendency to experience fouling due to accumulation of material. The bubble cap tray has a riser or chimney fitted over each hole and a cap that covers the riser. The cap is mounted so that there is a space between the riser and the cap to allow for passage of the vapor. The vapor rises through the chimney and is directed downward by the cap finally discharging through the holes in the chimney and bubbling on the tray. The bubble cap tray is most advanced and expensive of the three trays and are highly effective in low liquid flow rate situations and minimizing leakage.

A “theoretical distillation stage” is a hypothetical zone in which two phases, such as the liquid and the vapor phases of a substance, establish an equilibrium with each other. The performance of many separation processes depend on having a series of theoretical distillation stages. The performance of a separation device, such as the vacuum distillation unit may be enhanced by providing an increased number of stages. In an embodiment, the separation medium includes a sufficient number of theoretical distillation stages to effectively remove at least one product from the fermentation broth.

The term “product depleted stream” refers to a stream having reduced weight proportion of products such as ethanol, acetone, isopropanol, and combinations thereof, after distillation of the fermentation broth through the “vacuum distillation unit” compared to weight proportion of the products in the fermentation broth before the distillation. In certain instances, the product depleted stream comprises less than 20% of the product contained in the fermentation broth, or less than 10% of the product contained in the fermentation broth, or less than 5% of the product contained in the fermentation broth, or less than 2.5% of the product contained in the fermentation broth, or less than 2% of the product contained in the fermentation broth, or less than 1% of the product contained in the fermentation broth. The product depleted stream further contains components including, but are not limited to, waste water, biomass, acetate, 2,3-butanediol, and unused nutrients.

The term Mechanical Vapor Recompression (MVR) system is intended to encompass an energy recovery device which can be used to recycle waste heat to improve thermodynamic efficiency. Typically, a compressed vapor is generated by the MVR from a vaporised liquid and further condensation thereof is utilized to generate a portion of heating duty required for vaporisation of the liquid. Using same MVR system thermodynamically integrated with the vacuum distillation unit helps to maintain same mass flow rate at its overhead across all product streams i.e., ethanol, acetone, isopropanol, or combinations thereof handled by the vacuum distillation unit.

The term “rectification unit” is intended to encompass a device used downstream of the vacuum distillation unit to facilitate removing excess water and/or by-products from the vacuum distillation unit output. The rectification unit generally contains a greater number of theoretical distillation stages compared to the vacuum distillation unit. Further, the rectification unit includes multiple draw points to remove undesired products for example, C3-C4 alcohols, which may accumulate during product recovery process.

The term “drying unit” is intended to encompass a device such as a vessel or unit containing suitable adsorbent materials to adsorb excess water from the output stream of the rectification unit. Materials which can adsorb water that include, but are not limited to, aluminas, silicas, and molecular sieves such as synthetic or naturally occurring zeolites. Alternatively, the drying unit can comprise a polymeric membrane that can selectively allow a portion of one component from the output stream, for example, water to flow through to generate a permeate stream and does not allow a portion of other components of the output stream, for example, ethanol, acetone, isopropanol, or combinations thereof to flow thorough the membrane to generate a retentate stream or vice versa.

The term “ethanol-acetone separation unit” is intended to encompass a device to separate acetone and ethanol using fractional distillation. Boiling point of acetone is about 57° C. and of ethanol is about 78° C. When the ethanol-acetone mixture is boiled, the acetone is separated from the mixture during condensation, since the boiling point of the acetone is lower than that of the ethanol. The acetone can be collected from the ethanol-acetone separation unit overhead. Multiple distillation stages may be performed to improve purity of the separated acetone and ethanol.

The term “extractive distillation unit” is intended to encompass a device for distilling components with low relative volatilities, such as ethanol and isopropanol, through use of the addition of a third component, the extractive distillation agent, to modify the relative volatility of the components. To recover the extractive distillation agent, at least one separation column is utilized downstream of the extractive distillation unit.

The term “extractive distillation agent” is intended to encompass any component capable of modifying the relative volatility of the products. In an embodiment, the extractive distillation agent is capable of modifying the relative volatility of close boiling point products such as ethanol and isopropanol, to enable separation thereof. In addition to modifying the relative volatility, the extractive distillation agent may also have a high boiling point difference between the close boiling point products such as the ethanol and/or the isopropanol.

### Description

In some embodiments, feed gas stream for the disclosure may be obtained from industrial process selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, industrial processing of geological reservoirs, processing fossil resources such as natural gas coal and oil, or any combination thereof. Examples of specific processing steps within an industrial process include catalyst regeneration, fluid catalyst cracking, and catalyst regeneration. Air separation and direct air capture are other suitable industrial processes. Some examples in steel and ferroalloy manufacturing include, blast furnace gas, basic oxygen furnace gas, coke oven gas, direct reduction of iron furnace top-gas, and residual gas from smelting iron. In these embodiments, the substrate and/or the C1-carbon source may be captured from the industrial process before emitted into the atmosphere, using any known method.

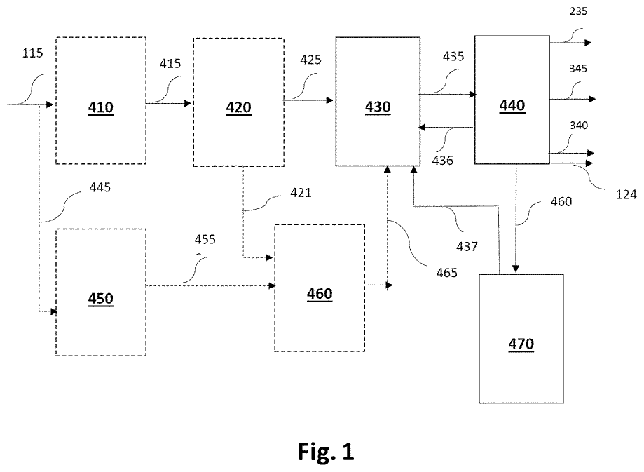

FIG. 1 shows a flow diagram for the production and the separation of products from a C1-containing feed gas stream according to one embodiment of the disclosure. The fermentation bioreactor 430 receives a first portion of the C1 containing feed gas stream from line 115. Optionally, the feed gas stream in the line 115 can be fed to a compressor 410 producing a compressed feed gas stream 415 which can optionally be passed to a contaminant removal reactor 420 producing a treated feed gas stream 425. The treated feed gas stream 425 passes through the fermentation bioreactor 430. The contaminant removal reactor 420 generally removes contaminants from the feed gas stream 115 which may be harmful to the C1 fixing microorganisms contained in the fermentation bioreactor 430. In some embodiment, the contaminant removal reactor 420 may include a deoxygenation catalyst, for example, a copper catalyst bed to remove oxygen.

A portion of the C1-containing feed gas stream delivered via a conduit 445 is optionally compressed by a second compressor 450 to produce a second compressed gas delivered via a conduit 455 to an inoculator reactor 460. The inoculator reactor 460 initiates cell growth of one or more microorganism to produce an inoculum. The fermentation bioreactor 430 receives the inoculum through a conduit 465. In some embodiment, the inoculator reactor 460 optionally receives compressed and treated gas from the contaminant removal reactor 420 directly through a conduit 421 which is further transferred to the fermentation bioreactor 430 via the conduit 465.

The fermentation bioreactor 430 comprises at least one Cl fixing microorganism in a liquid nutrient medium which ferments the C1 containing feed gas stream 115 to provide a fermentation broth 435 comprising fermentation products. The fermentation broth 435 comprises at least one of a first product stream comprising ethanol and water or a second product stream comprising ethanol, acetone, and water or a third product stream comprising ethanol, acetone, isopropanol, and water. Ethanol is generally produced as a native product during feed gas fermentation from acetaldehyde obtained from reductive synthesis of Acetyl-CoA produced during fermentation. However, the Acetyl-CoA is metabolised by a plurality of enzymes obtained from several genetically modified strains of the clostridium bacteria to yield acetone. These strains further improve selectivity of the acetone production by eliminating co-products for example, 3-hydroxybutyrate and 2,3-butanediol. Isopropanol is produced from acetone through enzymatic reduction performed by an enzyme secondary alcohol dehydrogenase. Not all of the acetone is converted to isopropanol. Therefore, during the isopropanol production, some of the excess acetone is recycled back to the fermentation bioreactor. Exemplary genetically modified microorganisms producing the isopropanol comprises culture of recombinant microorganisms capable to produce enzymes comprising an exogenous thiolase, an exogenous CoA transferase, and an exogenous decarboxylase. Other exemplary genetically modified microorganisms producing the acetone, the isopropanol and/or a precursor of the acetone and/or the isopropanol comprises culture of recombinant microorganisms capable to produce one or more enzymes selected from an Acetyl-CoA acetyltransferase, an Acetate CoA-transferase A, an Acetate CoA-transferase B, an Acetoacetate decarboxylase, and a α-ketoisovaleric acid decarboxylase. Genetically modified microorganisms capable to produce enzymes to yield the acetone/isopropanol are disclosed in granted patent U.S. Pat. No. 9,365,868 and published patent application WO 2012/115527 both of which are incorporated herein by reference.

The C1 fixing microorganism can be switched from a C1-fixing microorganism which produces the first product stream to one which produces the second product stream of ethanol, acetone, and water or the third product stream of ethanol, acetone, isopropanol, and water, or from a C1-fixing microorganism which produces the second product stream to one which produces the first product stream or the third product stream, or from a C1-fixing microorganism which produces the third product stream to one which produces the first product stream or the second product stream. One way to switch the C1-fixing microorganism in the fermentation bioreactor 430 involves the use of the inoculator reactor 460 in FIG. 1. The inoculator reactor 460 is shut down while the fermentation bioreactor 430 remains in operation. During the shutdown, the inoculator reactor vessel is drained, cleaned, and re-filled with fresh liquid nutrient medium and a different C1 fixing microorganism is introduced. The fermentation bioreactor 430 is shut down, drained, and cleaned. After the fermentation bioreactor 430 is cleaned and an inoculum is ready, the bioreactor 430 receives the inoculum via the conduit 465 and the new microorganism begins producing different fermentation products. The shut down and restart of the fermentation bioreactor 430 is coordinated with the inoculator reactor 460 restart to minimize production downtime.

The shared product recovery system 440 receives the fermentation broth 435 from the fermentation bioreactor 430. Output stream from the shared product recovery system 440 may comprise products having at least one of an enriched ethanol stream 235, an enriched acetone stream 340, an enriched isopropanol stream 345 or combinations thereof and an excess water stream 124. After separation and recovery of the products, a product depleted stream 436 is returned to the fermentation bioreactor 430. Excess water from the shared product recovery system 440 passes to a waste water treatment process 470. Purified water from the waste water treatment process 470 is recycled to the bioreactor 430 via a conduit 437.

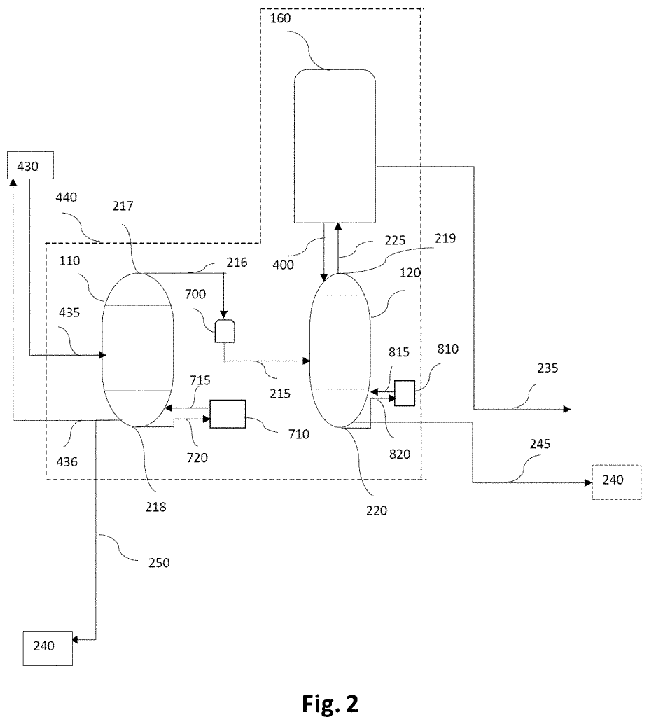

As shown in FIGS. 2, 3, and 4 the shared product recovery system 440 includes arranged device combinations of a vacuum distillation unit 110, a rectification unit 120, an acetone removal unit 130, a drying unit 160, an ethanol-acetone separation unit 140 and an extractive distillation unit 150 depending on the products to be recovered and separated from the fermentation broth. Providing such of the shared product recovery system 440 avoids building an individually customised facility to recover each of the products like ethanol, acetone, and isopropanol. Accordingly, the arranged device combinations in the shared product recovery system 440 reduces plant capital expenditure to a substantial extent.

In a first aspect of the disclosure, shown in FIG. 2, enriched anhydrous ethanol recovery from a fermentation broth comprising the first product stream comprising ethanol and water is disclosed. The shared product recovery system 440 according to the instant aspect, uses the vacuum distillation unit 110, the rectification unit 120, and the drying unit 160. The vacuum distillation unit 110 receives the fermentation broth 435 from the fermentation bioreactor 430. In an embodiment shown in FIG. 2, a reboiler 710 is used in conjunction with the vacuum distillation unit 110. The reboiler 710 is provided so as to direct a vapor stream to the vacuum distillation unit 110. The vapor stream is obtained by vaporisation of the liquid at the bottom 218 of the vacuum distillation unit 110 which exits therefrom via a conduit 720. The vapor stream from the reboiler 710 is directed through a conduit 715 to the vacuum distillation unit 110. The vapor stream entering the vacuum distillation unit 110 rises upward therethrough. The vacuum distillation unit 110 defines at least one separation section having multiple distillation trays (not shown). The performance of the separation process at the vacuum distillation unit 110 depends on the number of theoretical distillation stages. The vacuum distillation unit 110 operates in more than about 3 distillation stages in one embodiment, more than about 4 distillation stages in another embodiment, more than about 5 distillation stages in yet another embodiment.

To ensure effective separation of chemical products from the fermentation broth, the vacuum distillation unit 110 is generally operated at various temperature and pressure ranges. In various embodiments, the temperature is between 30° C. to 35° C. or 35° C. to 40° C. or 40° C. to 45° C. or 45° C. to 50° C. or 30° C. to 50° C. In various embodiments, the pressure at the bottom 218 of the vacuum distillation unit 110 is generally between 6 kPa(a) to 8 kPa(a) or 8 kPa(a) to 10 kPa(a) or 6 kPa(a) to 10 kPa(a). In various embodiments, the pressure at the overhead 217 of the vacuum distillation unit 110 is generally between 3 kPa(a) to 5 kPa(a) or 5 kPa(a) to 7 kPa(a) or 7 kPa(a) to 8 kPa(a) or 3 kPa(a) to 8 kPa(a).

The fermentation broth 435 comprising the first product stream comprising ethanol and water after passing through the vacuum distillation unit 110 produces an enriched ethanol stream 215 and a product depleted stream 436 that is returned to the bioreactor 430. In one embodiment, at least a portion of the product depleted stream 436 comprising waste water passes through a waste water treatment process 240 via a conduit 250 to produce a purified water stream which is recycled to the fermentation bioreactor 430 (not shown). In general instances, ethanol concentration at the fermentation broth 435 is about 2 wt %. In various embodiments, ethanol concentration of the enriched ethanol stream 215 is generally improved at least 4 fold by weight or at least 6 fold by weight or at least 8 fold by weight or at least 12 fold by weight compared to the ethanol concentration at the fermentation broth 435. Further, some enriched product vapor such as enriched ethanol vapor at the vacuum distillation unit 110 overhead 217 is passed to a Mechanical Vapor Compression system (MVR) 700 via a conduit 216. Compression and condensation of the enriched product vapor from the vacuum distillation unit 110 overhead 217 is thermodynamically beneficial to generate a substantial portion of the heating duty required by the vacuum distillation unit 110 which is generally at least 50% or at least 70% or at least 80% or at least 95%. Therefore, such compression and condensation of the enriched product vapor reduces overall steam consumption. As a result, the reboiler 710 duty is also optimized.

Enriched ethanol stream 215 sourced from the vacuum distillation unit 110 overhead 217 via the MVR system 700 passes through the rectification unit 120. In an embodiment, the rectification unit 120 further comprises at least one separation section (not shown). The separation section may include a series of distillation trays and/or packing material to facilitate the removal of excess water and/or by-products from the enriched ethanol stream 215. In some embodiments, the rectification unit 120 operates with more than about 30 theoretical distillation stages. In an embodiment, shown in FIG. 2, a reboiler 810 is used by the rectification unit 120. The reboiler 810 directs a vapor stream to the rectification unit 120. The vapor stream is obtained by vaporisation of the liquid at the bottom 220 of the rectification unit 120, which exits the rectification unit 120 via a conduit 820. This vapor stream is directed through a conduit 815 from the reboiler 810 to the rectification unit 120.

The rectification unit 120 produces an overhead ethanol stream 225 and a bottom water stream 245 which is recycled to the fermentation bioreactor 430 (not shown) either directly or after being treated in the waste water treatment process 240. In general instances, ethanol concentration of the enriched ethanol stream 215 is about 14 wt %. In various embodiments, ethanol concentration of the overhead ethanol stream 225 is generally improved at least 3 fold by weight or at least 5 fold by weight or at least 7 fold by weight compared to the ethanol concentration of the enriched ethanol stream 215. In various embodiments, the temperature of the rectification unit 120 overhead 219 is generally between 100° C. to 110° C. or 110° C. to 120° C. or 120° C. to 130° C. or 110° C. to 130° C. In various embodiments, the pressure at the rectification unit 120 overhead 219 is generally between 300 kPa(a) to 400 kPa(a) or 400 kPa(a) to 500 kPa(a) or 500 kPa(a) to 550 kPa(a) or 550 kPa(a) to 650 kPa(a) or 650 kPa(a) to 800 kPa(a) or 800 kPa(a) to 900 kPa(a) or 900 kPa(a) to 1100 kPa(a). The temperature and the pressure at the overhead 219 of the rectification unit 120 may be used as a basis to obtain its other operating conditions e.g., the bottom 220 temperature and pressure by using principles known in the art. The overhead ethanol stream 225 from the rectification unit 120 is transferred to a drying unit 160 to produce an anhydrous ethanol stream 235 and a purge stream 400. The drying unit 160 comprises two or more adsorbent beds housed in two or more vessels, through which the overhead ethanol stream 225 is flowed. When one of the adsorbent beds is saturated with water, the water has to be desorbed from the adsorbent bed to regenerate adsorption capacity. The saturated adsorbent bed is removed from operation and the overhead ethanol stream is switched to a fresh or regenerated adsorbent bed to dry the ethanol stream. The spent or saturated adsorbent bed is now regenerated by desorbing the water using a desorbent, such as the anhydrous ethanol, generated from the drying process. Regeneration conditions for desorbing water from an adsorbent bed are well known in the art. Once the adsorbent bed is regenerated, it is ready to be put into operation when the adsorbent bed currently in operation becomes saturated with water. Accordingly, the purge stream 400 having ethanol and water is produced and is withdrawn from the drying unit 160 and returned to the rectification unit 120 for further separation. In the embodiment where the drying unit 160 employs a polymeric membrane to remove water from the product stream, such as the overhead ethanol stream, only one adsorbent bed needs to be used. As stated, the polymeric membrane produces a retentate stream and a permeate stream. The non-product stream, whether the permeate or retentate stream, depending on the choice of membrane and separation conditions, is analogous to the purge stream 400 in the case of adsorbent using drying units, and is returned to the rectification unit 120.

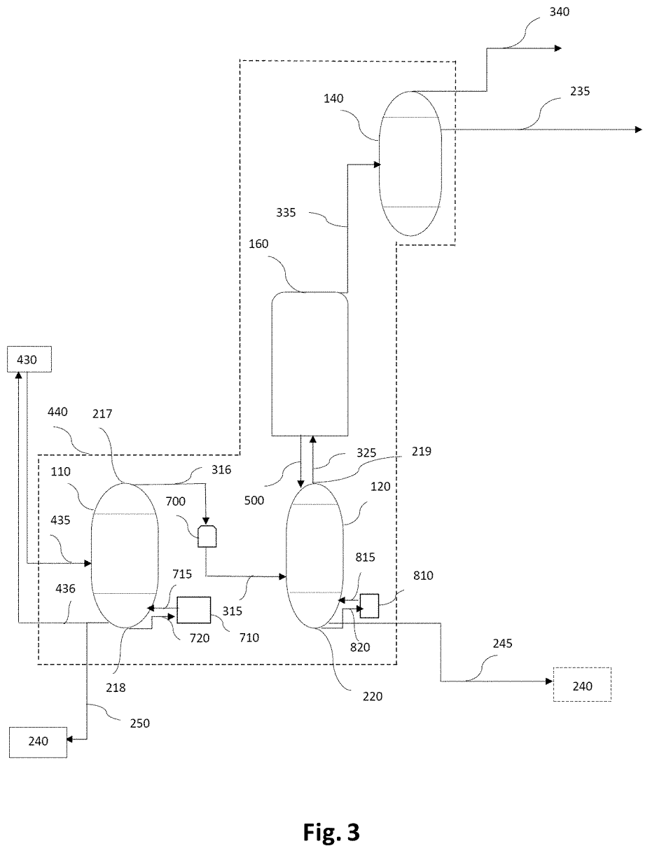

In a second aspect of the disclosure, shown in FIG. 3, enriched anhydrous acetone and ethanol recovery from a fermentation broth comprising the second product stream comprising acetone, ethanol and water is shown. The shared product recovery system 440 according to the instant aspect uses the vacuum distillation unit 110, the rectification unit 120, the drying unit 160 and the ethanol-acetone separation unit 140. The vacuum distillation unit 110 receives the fermentation broth 435 from the bioreactor 430. The fermentation broth 435 after passing through the vacuum distillation unit 110 produces a concentrated stream 315 enriched in acetone and ethanol and a product depleted stream 436. In one embodiment, at least a portion of the product depleted stream 436 comprising waste water passes through a waste water treatment process 240 via a conduit 250 to produce a purified water stream which is recycled to the fermentation bioreactor (not shown). In general instances, acetone and ethanol concentration at the fermentation broth 435 is about 2 wt %. In various embodiments, the acetone and the ethanol concentration in the concentrated stream 315 is generally improved at least 4 fold by weight or at least 6 fold by weight or at least 8 fold by weight or at least 12 fold by weight compared to the acetone and the ethanol concentration at the fermentation broth 435.

Concentrated stream 315 enriched in acetone and ethanol sourced from the vacuum distillation unit 110 overhead 217 via the MVR system 700 passes through the rectification unit 120. The rectification unit 120 produces an overhead stream 325 enriched in acetone and ethanol and a bottom water stream 245 which is recycled to the fermentation bioreactor 430 (not shown) either directly or after being treated in the waste water treatment process 240.

Constructional aspects of the vacuum distillation unit 110 and the rectification unit 120 including the MVR system 700 and the reboilers 710 and 810 are same as described in embodiments of FIG. 2. Further, process design parameters, for example, operating temperature and pressure of the vacuum distillation unit 110 and the rectification unit 120 in the second aspect of the disclosure is generally same as the first aspect of the disclosure. In general instances, acetone and ethanol concentration of the concentrated stream 315 enriched in acetone and ethanol is about 14 wt %. In various embodiments, the concentration of the acetone and the ethanol in the overhead stream 325 is generally improved at least 3 fold by weight or at least 5 fold by weight or at least 7 fold by weight compared to the concentrated stream 315 enriched in acetone and ethanol obtained from the vacuum distillation unit 110. The overhead stream 325 enriched in acetone and ethanol from the rectification unit 120 is passed to the drying unit 160. The drying unit 160 produces an anhydrous concentrated stream 335 enriched in acetone and ethanol and a purge stream 500 having acetone, ethanol and water. Mechanism for producing the purge stream 500 from the drying unit 160 using the adsorbent bed or the polymeric membrane is the same as the first aspect of the disclosure. The purge stream 500 is withdrawn from the drying unit 160 and returned to the rectification unit 120 for further separation.

The anhydrous concentrated stream 335 enriched in acetone and ethanol passes through the ethanol-acetone separation unit 140 which uses fractional distillation principle to produce an anhydrous acetone stream 340 from the ethanol-acetone separation unit 140 overhead and an anhydrous ethanol stream 235. The ethanol-acetone separation unit also operates in conjunction with the reboiler (not shown) as known in art.

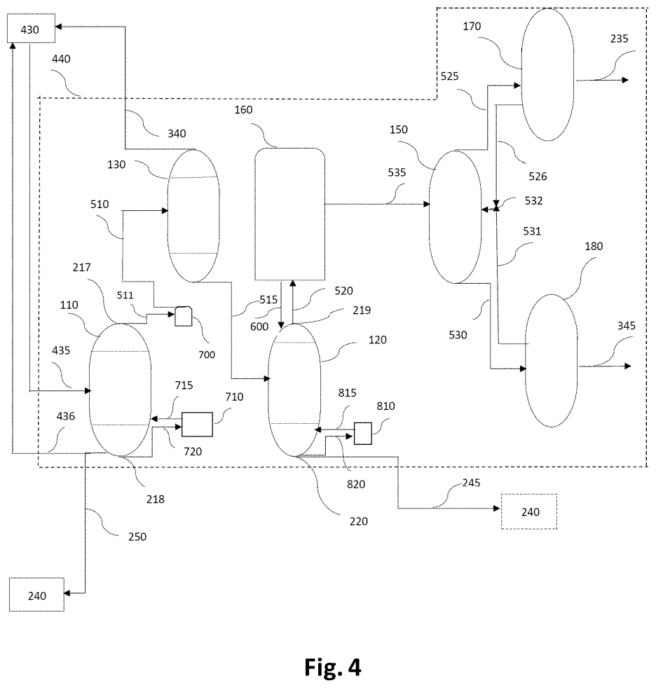

In a third aspect of the disclosure, shown in FIG. 4, an enriched anhydrous isopropanol stream and an anhydrous ethanol stream recovery from a fermentation broth comprising a third product stream comprising ethanol, acetone, isopropanol, and water is shown. The shared product recovery system 440 according to the instant aspect, uses the vacuum distillation unit 110, the acetone removal unit 130, the rectification unit 120, the drying unit 160, and the extractive distillation unit 150. Ethanol and isopropanol have close boiling points about 78.4° C. and about 82.4° C. respectively, thereby making the separation challenging. Therefore, extractive distillation has been found to effectively separate such close-boiling products. The vacuum distillation unit 110 receives the fermentation broth 435 from the bioreactor 430. The fermentation broth 435 after passing through the vacuum distillation unit 110 produces a concentrated stream 510 enriched in isopropanol, acetone, and ethanol and and a product depleted stream 436 that is returned to the bioreactor 430. In one embodiment, at least a portion of the product depleted stream 436 comprising waste water passes through a waste water treatment process 240 via a conduit 250 to produce a purified water stream which is recycled to the fermentation bioreactor (not shown). In general instances, isopropanol, acetone, and ethanol concentration at the fermentation broth 435 is about 2 wt %. In some embodiments, the concentration of the concentrated stream 510 is generally improved by at least 4 fold by weight or at least 6 fold by weight or at least 8 fold by weight or at least 12 fold by weight compared to isopropanol, acetone, and ethanol concentration in the fermentation broth 435.

The stream 510 enriched in isopropanol, acetone and ethanol sourced from the vacuum distillation unit 110 overhead 217 via the MVR unit 700 passes through the acetone removal unit 130. The acetone removal unit 130 produces a bottom stream 515 enriched in isopropanol and ethanol and an overhead stream 340 enriched in acetone. The overhead stream 340 enriched in acetone is recycled from the acetone removal unit 130 to the fermentation bioreactor 430 so that recycled acetone can be used for further isopropanol production. Further, the bottom stream 515 from the acetone removal unit 130 is passed through the rectification unit 120. The rectification unit 120 produces an overhead stream 520 enriched in ethanol and isopropanol and a bottom water stream 245 which is recycled to the fermentation bioreactor 430 (not shown) either directly or after being treated in the waste water treatment process 240. The overhead stream 520 enriched in ethanol and isopropanol is passed to the drying unit 160. The drying unit 160 produces an anhydrous concentrated stream 535 enriched in isopropanol and ethanol and a purge stream 600 having isopropanol, ethanol, and water. Mechanism for producing the purge stream 600 from the drying unit 160 using the adsorbent bed or the polymeric membrane is the same as the first aspect or the second aspect of the disclosure. The purge stream 600 is withdrawn from the drying unit 160 and returned to the rectification unit 120 for further separation.

An extractive distillation unit 150 receives the anhydrous concentrated stream 535 enriched in isopropanol and ethanol from the drying unit 160. The extractive distillation unit 150 enables distilling components with low relative volatilities, such as ethanol and isopropanol, through use of an extractive distillation agent. The extractive distillation agent works as a solvent by mixing with either the ethanol or the isopropanol present within the anhydrous concentrated stream 535. In an embodiment, the extractive distillation agent has a high affinity for one chemical product, either the ethanol or the isopropanol, and a low affinity for the other alternative product. A proper extractive distillation agent should not form an azeotrope with constituents of the anhydrous concentrated stream 535 enriched in ethanol and isopropanol and be capable of being separated from each of these products at subsequent separation columns during distillation.

An overhead stream 525 from the extractive distillation unit 150 passes to a separation column 170 to recover at least a portion of the anhydrous ethanol stream 235. A distillation bottom stream 530 from the extractive distillation unit 150 is passed to another separation column 180 to recover at least a portion of the anhydrous isopropanol stream 345. Distilled extractive distillation agents are recycled from the separation columns 170 and 180 through conduits 526 and 531 respectively and returned to the extractive distillation unit 150 through a conduit 532. Alternatively, in another embodiment (not shown in FIG. 4) the overhead stream 525 from the extractive distillation unit 150 is passed to the separation column 170 to recover at least a portion of the anhydrous isopropanol stream 345. The distillation bottom stream 530 from the extractive distillation unit 130 is passed to the another separation column 180 to recover at least a portion of the anhydrous ethanol stream 235. The extractive distillation unit 150 and the separation columns 170 and 180 also operate in conjunction with the reboilers (not shown) as known in art.

When at least a portion of the anhydrous ethanol stream 235 is recovered from the overhead stream 525 and at least a portion of the anhydrous isopropanol stream 345 is recovered from the distillation bottom stream 530, the extractive distillation agent may be selected from alpha-pinene, beta-pinene, methyl isobutyl ketone, limonene, alpha-phellandrene, alpha-terpinene, myrcene, carane, p-mentha-1,5-diene, butyl ether, 1-methoxy-2-propanol, n-butyl acetate, n-amyl acetate, benzyl acetate, ethylene glycol ethyl ether acetate, methyl acetoacetate, ethylene glycol diacetate, 2-butoxyethyl acetate, methyl butyrate, ethyl propionate, ethyl n-valerate, butyl benzoate, ethyl benzoate, pyridine, N,N-dimethyl aniline, o-sec.butyl phenol, 3-isopropyl phenol, 2,6-dimethyl phenol, o-tert.butyl phenol, 4-ethyl phenol, diethyl phthalate, diisooctyl phthalate, dimethyl adipate, glycerine triacetate, diethyl malonate, dimethyl glutarate, tetrahydrofuran, ethylene glycol phenyl ether, dipropylene glycol methyl ether acetate, diethylene glycol hexyl ether, propoxypropanol, butoxypropanol, p-xylene glycol dimethyl ether, diethylene glycol t-butyl ether methyl ether, triethylene glycol diacetate, anisole, phenetole, phenyl ether, 1,2-methylenedioxybenzene, isophorone, ethyl ethoxypropionate, tetraethyl ortho silicate, 2-hydroxyacetophenone, 1,1,1-trichloroethane, tetrachloroethylene, 2,2,2-trichloroethanol, m-dichlorebenzene, chlorobenzene, 2,6-dichlorotoluene, 1-chlorohexane, diethylene glycol, dimethyl sulfoxide, dimethylformamide, sulfolane, isophorone, 2-pyrrolidione, 1-methyl-2pyrrolindinone, isodecyl alcohol, cyclododecanol, benzyl alcohol, 1-dodecanol, tridecyl alcohol, phenethyl alcohol, cyclohexanol, cyclopentanol, 2-nitropropane, 1-nitropropane, nitro-ethane, nitromethane, 3-nitrotoluene, 2-nitrotoluene, triacetin, 3-nitro-o-xylene, 1,4-dioxane, isobutyl acetate, ethyl butyrate, isoamyl formate, methyl caproate, ethyl caproate, propyl caproate, 1-methoxy-2-propanol acetate, isobutyl isobutyrate, hexyl acetate, ethyl isobutyrate, propyl butyrate, isobutyl butyrate, isobornyl acetate, 1,3-dioxolane, nitrobenzene, butyl butyrate, 4-methyl-2-pentanone, and polyethylene glycol.

When at least a portion of the anhydrous isopropanol stream 345 is recovered from the overhead stream 525 and at least a portion of the anhydrous ethanol stream 235 is recovered from the distillation bottom stream 530 the extractive distillation agent may be selected from ethyl benzene, toluene, p-xylene, heptane, phenol, and 2-tert-butyl phenol.

All references, including publications, patent applications, and patents, cited herein are hereby incorporated by reference to the same extent as if each reference was individually and specifically indicated to be incorporated by reference and were set forth in its entirety herein. The reference to any prior art in this specification is not, and should not be taken as, an acknowledgement that that prior art forms part of the common general knowledge in the field of endeavour in any country.

Recitation of ranges of values herein are merely intended to serve as a shorthand method of referring individually to each separate value falling within the range, unless otherwise indicated herein, and each separate value is incorporated into the specification as if it were individually recited herein. For example, unless otherwise indicated, any concentration range, percentage range, ratio range, integer range, size range, or thickness range is to be understood to include the value of any integer within the recited range and, when appropriate, fractions thereof (such as one tenth and one hundredth of an integer). Unless otherwise indicated, ratios are molar ratios, and percentages are on a weight basis.

All methods described herein can be performed in any suitable order unless otherwise indicated herein or otherwise clearly contradicted by context. The use of any and all examples, or exemplary language (i.e., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein. Variations of those embodiments may become apparent to those of ordinary skill in the art upon reading the foregoing description, and employment of such variations as appropriate, is intended to be within the scope as the disclosure may be practiced otherwise than as specifically described herein. Accordingly, this disclosure includes all modifications and equivalents of the subject matter recited in the claims as permitted by applicable law. Moreover, any combination of the above described elements in all possible variations thereof is encompassed by the disclosure unless otherwise indicated herein or otherwise clearly contradicted by context.

Claims:

1. A process for producing and recovering at least one product from a fermentation process comprising;
a) introducing a C1-containing gas from a source to a fermentation bioreactor containing at least one C1-fixing microorganism, in a liquid nutrient medium, to produce a fermentation broth comprising at least one of a first product stream comprising ethanol and water or a second product stream comprising ethanol, acetone, and water or a third product stream comprising ethanol, acetone, isopropanol, and water; and
b) transferring the fermentation broth from the fermentation bioreactor to a shared product recovery system for selectively recovering at least one enriched product stream selected from an enriched ethanol stream, an enriched acetone stream, an enriched isopropanol stream or combinations thereof.

2. The process of claim 1, wherein the shared product recovery system comprises at least one of a vacuum distillation unit, a rectification unit, an acetone removal unit, a drying unit, an ethanol-acetone separation unit, an extractive distillation unit or combinations thereof.

3. The process of claim 1, wherein the C1-fixing microorganism is switched from a C1-fixing microorganism which produces the first product stream to one which produces the second product stream of ethanol, acetone, and water or the third product stream of ethanol, acetone, isopropanol, and water; or from a C1-fixing microorganism which produces the second product stream to one which produces the first product stream or the third product stream; or from a C1-fixing microorganism which produces the third product stream to one which produces the first product stream or the second product stream.

4. The process of claim 2, wherein the enriched ethanol stream is produced by passing the fermentation broth comprising the first product stream to the vacuum distillation unit operated at conditions to produce an enriched ethanol stream and a product depleted stream wherein the product depleted stream is returned to the fermentation bioreactor.

5. The process of claim 4, further comprising passing the enriched ethanol stream from the vacuum distillation unit to the rectification unit to produce an overhead ethanol stream and a bottom water stream wherein the bottom water stream is recycled to the fermentation bioreactor either directly or after being treated in a waste water treatment process.

6. The process of claim 5, further comprising passing the overhead ethanol stream from the rectification unit to the drying unit to produce an anhydrous ethanol stream and a purge stream wherein the purge stream is returned to the rectification unit.

7. The process of claim 4, further comprising passing at least a portion of the product depleted stream comprising waste water to the waste water treatment process to produce a purified water stream which is recycled to the fermentation bioreactor.

8. The process of claim 2, wherein the enriched acetone stream is produced by passing the fermentation broth comprising the second product stream to the vacuum distillation unit operated at conditions to produce a concentrated stream enriched in acetone and ethanol and a product depleted stream wherein the product depleted stream is returned to the fermentation bioreactor.

9. The process of claim 8, further comprising passing the concentrated stream from the vacuum distillation unit to the rectification unit to produce an overhead stream enriched in acetone and ethanol, and a bottom water stream wherein the bottom water stream is recycled to the fermentation bioreactor either directly or after being treated in a waste water treatment process.

10. The process of claim 9, further comprising passing the overhead stream enriched in acetone and ethanol from the rectification unit to the drying unit to produce an anhydrous concentrated stream enriched in acetone and ethanol and a purge stream wherein the purge stream is returned to the rectification unit.

11. The process of claim 10, further comprising passing the anhydrous concentrated stream enriched in acetone and ethanol from the drying unit to the ethanol-acetone separation unit to produce an anhydrous acetone stream and an anhydrous ethanol stream.

12. The process of claim 8, further comprising passing at least a portion of the product depleted stream comprising waste water to the waste water treatment process to produce a purified water stream which is recycled to the fermentation bioreactor.

13. The process of claim 2, wherein the enriched isopropanol stream is produced by passing the fermentation broth comprising the third product stream to the vacuum distillation unit to produce a concentrated stream enriched in isopropanol, acetone, and ethanol and a product depleted stream wherein the product depleted stream is returned to the fermentation bioreactor.

14. The process of claim 13, further comprising passing the concentrated stream enriched in isopropanol, acetone, and ethanol from the vacuum distillation unit to the acetone removal unit to produce a bottom stream enriched in isopropanol and ethanol and an overhead stream enriched in acetone.

15. The process of claim 14, further comprising recycling the overhead stream from the acetone removal unit to the fermentation bioreactor to produce isopropanol.

16. The process of claim 14, further comprising passing the bottom stream enriched in isopropanol, and ethanol from the acetone removal unit to the rectification unit to produce an overhead stream enriched in isopropanol and ethanol, and a bottom water stream wherein the bottom water stream is recycled to the fermentation bioreactor either directly or after being treated in a waste water treatment process.

17. The process of claim 16, further comprising passing the overhead stream enriched in isopropanol and ethanol from the rectification unit to the drying unit to produce an anhydrous concentrated stream enriched in isopropanol and ethanol and a purge stream wherein the purge stream is returned to the rectification unit.

18. The process of claim 13, further comprising passing at least a portion of the product depleted stream comprising waste water to the waste water treatment process to produce a purified water stream which is recycled to the fermentation bioreactor.

19. The process of claim 17, further comprising passing the anhydrous concentrated stream enriched in isopropanol and ethanol from the drying unit to the extractive distillation unit to distill the anhydrous concentrated stream in presence of least one extractive distillation agent to obtain an overhead stream and a distillation bottom stream, wherein:
i. at least a portion of anhydrous ethanol is recovered in the overhead stream and at least a portion of anhydrous isopropanol is recovered in the distillation bottom stream; or
ii. at least a portion of anhydrous isopropanol is recovered in the overhead stream and at least a portion of anhydrous ethanol is recovered in the distillation bottom stream.

20. The process of claim 19,
wherein at least a portion of the anhydrous ethanol is recovered in the overhead stream and at least a portion of the anhydrous isopropanol is recovered in the distillation bottom stream; and
wherein the extractive distillation agent comprises at least one compound selected from alpha-pinene, beta-pinene, methyl isobutyl ketone, limonene, alpha- phellandrene, alpha-terpinene, myrcene, carane, p-mentha-1,5-diene, butyl ether, 1-methoxy-2-propanol, n-butyl acetate, n-amyl acetate, benzyl acetate, ethylene glycol ethyl ether acetate, methyl acetoacetate, ethylene glycol diacetate, 2-butoxyethyl acetate, methyl butyrate, ethyl propionate, ethyl n-valerate, butyl benzoate, ethyl benzoate, pyridine, N,N-dimethyl aniline, o-sec.butyl phenol, 3-isopropyl phenol, 2,6-dimethyl phenol, o-tert.butyl phenol, 4-ethyl phenol, diethyl phthalate, diisooctyl phthalate, dimethyl adipate, glycerine triacetate, diethyl malonate, dimethyl glutarate, tetrahydro furan, ethylene glycol phenyl ether, dipropylene glycol methyl ether acetate, diethylene glycol hexyl ether, propoxypropanol, butoxypropanol, p-xylene glycol dimethyl ether, diethylene glycol t-butyl ether methyl ether, triethylene glycol diacetate, anisole, phenetole, phenyl ether, 1,2-methylenedioxybenzene, isophorone, ethyl-3-ethoxypropionate, tetraethylorthosilicate, 2-hydroxyacetophenone, 1,1,1-trichloroethane, tetrachloroethylene, 2,2,2-trichloroethanol, m-dichlorebenzene, chlorobenzene, 2,6-dichlorotoluene, 1-chlorohexane, diethylene glycol, dimethyl sulfoxide, dimethylformamide, sulfolane, isophorone, 2-pyrrolidione, 1-methyl-2pyrrolindinone, isodecyl alcohol, cyclododecanol, benzyl alcohol, 1-dodecanol, tridecyl alcohol, phenethyl alcohol, cyclohexanol, cyclopentanol, 2-nitropropane, 1-nitropropane, nitro-ethane, nitromethane, 3-nitrotoluene, 2-nitrotoluene, triacetin, 3-nitro-o-xylene, 1,4-dioxane, isobutyl acetate, ethyl butyrate, isoamyl formate, methyl caproate, ethyl caproate, propyl caproate, 1-methoxy-2-propanol acetate, isobutyl isobutyrate, hexyl acetate, ethyl isobutyrate, propyl butyrate, isobutyl butyrate, isobornyl acetate, 1,3-dioxolane, nitrobenzene, butyl butyrate, 4-methyl-2-pentanone, and polyethylene glycol 400.

21. The process of claim 19,
wherein at least a portion of the anhydrous isopropanol is recovered in the overhead stream and at least a portion of the anhydrous ethanol is recovered in the distillation bottom stream; and
wherein the extractive distillation agent comprises at least one compound selected from ethyl benzene, toluene, p-xylene, heptane, phenol, and 2-tert-butyl phenol.

22. The process of claim 1, where the C1-fixing microorganism is at least one carboxydotrophic bacteria.

23. The process of claim 22, wherein the carboxydotrophic bacteria is selected from Clostridium autoethanogenum, Clostridium ljungdahlii, Clostridium ragsdalei, and mixtures thereof.

24. A system to recover at least one product from a gas fermentation process comprising;
(a) a C1-gas fermentation bioreactor in fluid communication with a vacuum distillation unit configured to produce an enriched ethanol stream and a product depleted stream from a first product stream comprising ethanol and water; and
(b) a rectification unit in fluid communication with the vacuum distillation unit, the rectification unit being configured to produce an overhead ethanol stream and a bottom water stream.

25. The system of claim 24, wherein a drying unit is in fluid communication with the rectification unit, the drying unit being configured to produce an anhydrous ethanol stream and a purge stream.

26. The system of claim 24, wherein the vacuum distillation unit is configured to thermodynamically integrate with a mechanical vapor recompression system.

27. A system to recover at least one product from a gas fermentation process comprising;
(a) a C1-gas fermentation bioreactor in fluid communication with a vacuum distillation unit configured to produce a concentrated stream enriched in acetone and ethanol and a product depleted stream from a second product stream comprising ethanol, acetone, and water;
(b) a rectification unit in fluid communication with the vacuum distillation unit, the rectification unit being configured to produce an overhead stream enriched in acetone and ethanol and a bottom water stream;
(c) a drying unit in fluid communication with the rectification unit, the drying unit being configured to produce an anhydrous concentrated stream enriched in acetone and ethanol and a purge stream; and
(d) an ethanol-acetone separation unit in fluid communication with the drying unit, the ethanol-acetone separation unit being configured to produce an anhydrous acetone stream and an anhydrous ethanol stream.

28. The system of claim 27, wherein the vacuum distillation unit is configured to thermodynamically integrate with a mechanical vapor recompression system.

29. A system to recover at least one product from a gas fermentation process comprising;
(a) a C1-gas fermentation bioreactor in fluid communication with a vacuum distillation unit configured to produce a concentrated stream enriched in isopropanol, acetone, and ethanol and a product depleted stream from a third product stream comprising ethanol, acetone, isopropanol, and water;
(b) an acetone removal unit in fluid communication with the vacuum distillation unit, the acetone removal unit being configured to produce a bottom stream enriched in isopropanol and ethanol and an overhead stream rich in acetone;
(c) a rectification unit in fluid communication with the acetone removal unit, the rectification unit being configured to produce an overhead stream enriched in isopropanol and ethanol and a bottom water stream from the bottom stream enriched in isopropanol and ethanol;
(d) a drying unit in fluid communication with the rectification unit, the drying unit being configured to produce an anhydrous concentrated stream enriched in isopropanol and ethanol and a purge stream; and
(e) an extractive distillation unit in fluid communication with the drying unit, the extractive distillation unit being configured to obtain an overhead stream and a distillation bottom stream from distillation of the anhydrous concentrated stream enriched in isopropanol and ethanol in presence of at least one extractive distillation agent.

30. The system of claim 29, wherein the extractive distillation unit is in fluid communication with a separation column and another separation column, being configured to recover;
at least a portion of anhydrous ethanol from the overhead stream and at least a portion of anhydrous isopropanol from the distillation bottom stream; or
(ii) at least a portion of anhydrous isopropanol from the overhead stream and at least a portion of anhydrous ethanol from the distillation bottom stream.

31. The system of claim 29, wherein the acetone removal unit is in further fluid communication with the fermentation bioreactor, the acetone removal unit being configured to recycle the overhead stream enriched in acetone to the fermentation bioreactor.

32. The system of claim 29, wherein the vacuum distillation unit is configured to thermodynamically integrate with a mechanical vapor recompression system.
